(19) United States
(12) Patent Application Publication
(10) Pub. No.: US 2010/0144790 A1
(43) Pub. Date: Jun. 10, 2010

(54) Use of Thalidomide for Alzheimers Disease Treatment and Prevention

(76) Inventors: Yong Shen, Scottsdale, AZ (US); Marwan N. SABBAGH, Scottsdale, AZ (US)

Correspondence Address: JENNINGS, STROUSS & SALMON, P.L.C., 201 E. WASHINGTON ST., 11TH FLOOR, PHOENIX, AZ, 85004, US

(21) Appl. No.: 12/631,670

(22) Filed: Dec. 4, 2009

Related U.S. Application Data
(60) Provisional application No. 61/119,940, filed on Dec. 4, 2008.

(51) Int. Cl. A61K 31/454; A61P 25/00; A61P 25/28

(52) U.S. Cl. 514/323

(57) ABSTRACT

β-secretase (BACE) is a biomarker for MCI and Alzheimer's disease. BACE1 protein level and the enzymatic activity level increase in the brain, and in the CSF in AD and MCI patients. Increased BACE1 levels in CSF are related to neuronal death and synaptic damages, which contribute to AD-related cognitive deficits. BACE1 inhibitors may be used as therapeutic compounds for clinical use for the disease treatment and prevention. Use of Thalidomide, an effective BACE1 inhibitor, reduces the BACE1 enzymetic activity and protein levels, and the Aβ production that marks the neurodegenerative progression associated with AD.

### CROSS REFERENCE TO RELATED APPLICATION

This Application claims the benefit of U.S. Provisional Patent Application No. 61/119,940, filed on Dec. 4, 2008.

### BACKGROUND OF THE INVENTION

Alzheimer's disease (AD) is the most common form of dementia. As of September 2009, this number is reported to be 35 million-plus worldwide. The prevalence of Alzheimer's is thought to reach approximately 107 million people by 2050. The cause and progression of Alzheimer's disease are not well understood. The progressive formation of amyloid plaques and vascular deposits of amyloid β-peptide has long been considered the pathological hallmark of Alzheimer's disease. Only a few medications have currently been approved by FDA for treating the cognitive manifestations of AD, but none has indication of delaying or halting the progression of the disease.

Thalidomide has been extensively used in humans, and inconsistent results have been observed for treating different diseases. Thalidomide is a sedative-hypnotic, and multiple myeloma medication. Thalidomide was chiefly sold and prescribed during the late 1950s and early 1960s to pregnant women, as an antiemetic to combat hyperemesis gravidarium and as a sleep aid. Because of its significant teratogenicity, it has not been used since the '60s except for orphan drug use indications. Subsequent research has shown that it is effective in multiple myeloma, and it was approved by the FDA for use in this malignancy. The FDA has also since approved the drug's use in the treatment of erythema nodosum leprosum. Thalidomide, in doses of up to 1200 mg daily, has been shown to extend life expectancy of patients with glioblastoma multiforme when administered with XRT and other chemotherapeutic agents. However, more recently, Thalidomide was not shown to increase life expectancy or improve survival in patients with multiple brain metastases. Thalidomide continues to be available for the prescription of malignancies and other conditions but with severe restrictions and oversight from the FDA, and clinicians are required to undergo certification prior to prescribing Thalidomide through the manufacturer Celgene.

### BRIEF SUMMARY OF THE INVENTION

The present invention provides a method of treatment of cognitive deficit diseases, which include Alzheimer's disease, MCI, and Alzheimer's related dementia in human or other animals, as well as other BACE1-related diseases in which BACE1 protein level or enzymatic activity level is increased compared to normal control. Thalidomide, which is an inhibitor of BACE1, is disclosed as a treatment for BACE1-related diseases.

The above may be achieved using methods involving treating or preventing of dementia, such as Alzheimer's disease or MCI, with a therapeutically effective amount of a pharmaceutical composition that includes Thalidomide or a pharmaceutically acceptable prodrug, salt, solvate, hydrate, or clathrate thereof.

Aspects and applications of the invention presented here are described below in the drawings and detailed description of the invention.

### BRIEF DESCRIPTION OF THE FIGURES

A more complete understanding of the present invention may be derived by referring to the detailed description when considered in connection with the following illustrative figures.

FIG. 1 shows the BACE1 protein expression level is increased in brain of AD patients in comparison to normal control. FIG. 1a used western blotting for the measurement; and FIG. 1b used BACE1 ELISA. All brain samples are from individuals with the diagnosis of sporadic AD and the BACE1 levels were normalized by actin.

FIG. 2 used the Fluorescent labeled APPsw peptide to detect the BACE1 enzymatic activity level. FIG. 2 shows that the BACE1 enzymatic activity is increased in brain of AD patients sporadic AD brains. FIG. 2A depicts that BACE1 enzymatic activity is increased higher in AD brains than in ND brains. The data is statistically significant and the correlation was at **p<0.001. FIG. 2B shows the immunoprecipitation result depicting that the β-secretase cleavage product, C99, was more in AD brains, although the full-length APP remained at the same level in comparison to the normal control.

FIG. 3 shows the Western Blotting depicting that the BACE1 protein expression level increases in CSF of AD and MCI patient. Two different antibodies were used to detect BACE1 protein: anti-BACE1 N-terminus polyclonal antibody and anti-BACE1 C-terminus polyclonal antibody. Both antibodies detected the −70 KD BACE1 and −60 KD BACE1 protein bands. Both the −70 KD band and −60 KD band of BACE1 were significantly higher in MCI than both the AD and CN CSF samples. However, there is no statistically significant difference between AD and CN CSF BACE1 protein levels.

FIG. 4 shows the result of CSF BACE1 Deglycosylation assay for the level of BACE1 protein level. Both bands of BACE1 protein could be deglycosylated by PNGase F and resulted in a single 50 KD species, which corresponds to the size of the deglycosylated form of BACE1 protein, shows that both mature and immature forms of BACE1 protein exist in CSF.

FIG. 5 shows that the method of two sandwich ELISAs was established to measure BACE1 protein level. Left: Capture antibody is anti-BACE1 N-terminus antibody SECB2, the detection antibody is biotinylated anti-BACE1 polyclonal antibody SECB1. Right: Capture antibody is anti-BACE1 polyclonal antibody B280 and the detection antibody is anti-BACE1 monoclonal antibody.

FIG. 6 shows that the total Aβ₁₋ₓ and the skeleton neuronal protein, tau, levels are correlated in CSF of patients with MCI and AD. FIG. 6A establishes the method of measuring the CSF total Aβ₁₋ₓ concentration by ELISA. FIG. 6B shows the total Aβ₁₋ₓ levels in controls, patients with MCI and AD. The mean±SD total Aβ levels in controls is 42.03±37.55 ng/mL, in patients with MCI is 100.03±73.50 ng/mL, and in patients with AD is 63.37±33.88 ng/mL. FIG. 6C shows the total tau levels in CSF which were measured using a sandwich ELISA. The mean±SD total tau level in controls is 356.4±144.2 pg/mL, in patients with MCI is 623.18±474.5 pg/mL, and in patients with AD is 645.16±349.7 pg/mL. Horizontal lines represent medians; boxes, 25th and 75th percentile boundaries; and error bars, range. **P<0.001; *P<0.01.

FIG. 7 depicts the result of double staining in which the primary antibodies was applied against calbindin, and it shows the reduction of calcium dependent protein, calbindin, in the mossy fibers and cellular body of the dentate gyrus in 24-month-old APP23 mice, which is a sign of synaptic damage associated with AD.

FIG. 8 depicts the result of using the staining method in which the rabbit anti-synaptophysin was applied to show that the increased BACE1 protein expression level is colocalized with synapse injury in APP transgenic mouse brains in comparison to the wildtype. Decreased numbers of synapses and increased BACE1 expression were observed. The arrows show the degenerated and aggregated synapses and BACE1 expressions in one plaque in the cortex of APP23 mice.

FIG. 9 shows that the BACE1 enzymatic activity level was increased in brains of APP23 transgenic mice. BACE1 enzyme activity was evaluated using APP23 mouse brain enzymatic extracts incubated with fluorescently labeled peptides bearing the APP β-cleavage site from the Swedish mutant APP fragment (APPsw). Fluorescently labeled peptides were detectable only upon cleavage by BACE1. APP mouse brains (n=5) showed significantly higher BACE1 activity in APP23 mice at 24 months compared to APP23 mice of younger age. All quantitations were the mean±SD, of at least 3 independent tests. Significant correlation was at **P<0.001.

FIG. 10 shows that BACE1 protein expression was increased in brains of APP23 transgenic mice at ages of 12 and 24 months old by BACE1 ELISA, in comparison to wildtype mice of the same age. Brain homogenate samples from the frontal cortex (n=5) of APP23 mice, were quantitatively assayed using BACE1 ELISA. All quantitations are the mean±SD of at least 3 independent measurements. Significant correlation was at **P<0.001.

FIG. 11 shows double staining of monoclonal antibody against BACE1 in the hippocampal CA3 of 24 month old mice. It shows that BACE1 increased protein expression co-localizes with AMPA receptor, GluR1, in aged APP23 mice. GluR1 (Glutamate receptors subtype 1) were primarily expressed on the cellular plasmal membrane in the hippocampal CA3 neuron cells of 24 month old mice. The arrows in WT mice show GluR1 expression on the cell membrane of the CA3 neurons but decreased expression on the neuronal membrane of the CA3 region in APP23 mice at the 24-month-old. The BACE1 expressed primarily on the membrane of the neurons as well, and the expression increases in APP23 transgenic mice at 24 months in comparison to wildtype (WT).

FIG. 12 shows rabbit polyclonal antibody against NMDA receptor subtype I, NR1. The immunologically excited glutamate receptor shows that BACE1 co-localizes with NMDA receptor, NR1, in aged APP23 mice primarily on the membrane of the neurons. The arrows show that dotted clustering NR1 (NMDA receptor subtype 1) expression was increased in the hippocampal CA3 of the 24 month old APP23 transgenic mice.

FIG. 13 depicts the 6E10 immunostaining to determine the plaque number in the APP23 mice after Thalidomide injection. It shows that the level of Aβ deposition was reduced in the brain of APP23 mice after Thalidomide treatment for one month at a dosage of 100 mg/kg. The plaque number in the APP23 mice decreases compared to the APP23 mice without Thalidomide treatment in the frontal cortex at the same age. At 12 months of age, APP23 mice had extensive Aβ plaques, the count of which was 84.6/section. However, few Aβ plaques were detectable in the frontal cortex of APP23 mice after treatment with Thalidomide for one month, the count of which is 3.91/section.

FIG. 14 shows that the BACE1 enzymatic activity level was reduced in the brain of APP23 mice after Thalidomide treatment. The BACE1 activity level was measured using aged (12 months old) APP23 transgenic mouse frontal cortex brain enzymatic crude extracts, which was then incubated with fluorescent-labeled peptide-bearing β-sites from APP protein. The BACE1 activity from the APP23 mouse brain tissue after Thalidomide injection for one month at a dose of 100 mg/kg decreases compared to the APP23 mice without Thalidomide treatment in the frontal cortex at the same age.

FIG. 15 provides the descriptive statistics of age, gender, MMSE, and CSF-based assessment of CSF samples from a study group of 80 AD patients, 59 MCI patients and 69 healthy controls (HC). The age, gender and MMSE test result data were associated with the values of CSF BACE1 protein level, CSF BACE1 enzymatic activity level, CSF total Aβ level and CSF total tau level. The values of CSF BACE1 protein level, CSF BACE1 activity, CSF total Aβ and CSF total tau levels are shown as [mean±standard deviation]. The data compilation shows that the increased BACE1 protein level, BACE1 enzymatic activity level, total Aβ level, and total tau level correlated with higher risk of AD, when AD patient group were compared to CN group. The correlated higher risk of MCI was also observed when the MCI patient group was compared to the CN group. But no elevated risk of AD in comparison to MCI patients was observed based on the level of tau protein level. Label 1 represents “Statistically significant in comparison to AD and MCI, p<0.001”; Label 2 represents “Statistically significant in comparison to CN, p<0.001”; Label 3 represents “Statistically significant in comparison to MCI, p<0.001”; Label 4 represents “Statistically significant in comparison to MCI, p<0.001”; Label 5 represents “Statistically significant in comparison to AD and MCI, p<0.01”; Label 6 represents “Statistically significant in comparison to AD and MCI, p<0.01.”

FIG. 16 provides the relative risk ratios for group comparisons based on uni-variate and multi-variate prediction models each controlled for age among the AD patient group, MCI patient group and the Healthy Control group. The mean concentration of BACE1 in the CSF differed significantly between groups (F=23.4; df=2, 185; P<0.001). BACE1 protein levels in the CSF of MCI patients were significantly higher than in AD patients (P<0.001) and CN (P<0.001), but the difference in BACE1 between MCI and AD was not significant (P=0.79). Logistic regression analysis showed that increased levels of BACE1 were associated with increased risk of MCI when compared to CN or AD, and of AD when compared to CN (for risk ratios, 95% Cls, and classification accuracy).

Elements and acts in the figures are illustrated for simplicity and have not necessarily been rendered according to any particular sequence or embodiment.

### DETAILED DESCRIPTION OF THE INVENTION

In the following description, and for the purposes of explanation, numerous specific details are set forth in order to provide a thorough understanding of the various aspects of the invention. It will be understood, however, by those skilled in the relevant arts, that the present invention may be practiced without these specific details. In other instances, known structures and devices are shown or discussed more generally in order to avoid obscuring the invention.

This invention encompasses pharmaceutical dosage forms of Thalidomide and pharmaceutically acceptable prodrugs, salts, solvates, hydrates, and clathrates thereof. The invention further encompasses a novel method of treating or preventing diseases and conditions such as, but not limited to, Alzheimer's disease, MCI, Alzheimer's related dementia, using Thalidomide and pharmaceutically acceptable prodrugs, salts, solvates, hydrates, and clathrates thereof. Only for the purpose of illustration but not to be limited to the particular mechanism, the invention encompasses a novel target, BACE1, for Alzheimer's disease; and the application of Thalidomide as BACE1 inhibitor to reduce BACE1 enzymatic activity and protein levels, and thus the deposit of amyloid β-peptide, which is the pathological hallmark of Alzheimer's disease.

Pharmaceutical compositions and dosage forms of the invention contain a prophylactically or therapeutically effective amount of an active ingredient (i.e., Thalidomide or a pharmaceutically acceptable prodrug, salt, solvate, hydrate, or clathrate thereof) and an excipient. Preferred dosage forms are suitable for oral administration, and can be coated to reduce or avoid degradation of the active ingredient within the gastrointestinal tract.

Pharmaceutical packs or kits which comprise pharmaceutical compositions or dosage forms disclosed herein are also encompassed by the present invention. An example of a kit comprises notice in the form prescribed by a governmental agency regulating the manufacture, use or sale of pharmaceuticals or biological products, which notice reflects approval by the agency of manufacture, use or sale for human administration.

Amyloid β peptide (Aβ) a major protein component of the senile plaque, is generated from amyloid precursor protein (APP) by enzymatic digestion involving (β-secretase (BACE1) and γ-secretase activities. However, the mechanisms of Aβ accumulation in the majority of AD patients (sporadic AD) remain unclear. BACE1 is a transmembrane aspartyl protease and overexpression of BACE1 in transfected cells increases the amount of C99 and C89, which are both BACE1-cleavage products and are precursors for Aβ formation.

Aβ is the pathological hallmark of Alzheimer's disease. The progressive formation of amyloid plaques and vascular deposits consisting of the 4-KD amyloid β-peptide (Aβ) has long been considered the pathological hallmark of Alzheimer's disease (AD). Under normal conditions, small amounts of soluble Aβ40 and Aβ42 circulate in the bloodstream. For individuals with the deterministic mutations for sporadic AD in presenilin 1 (PS1), PS2, or amyloid precursor protein (APP) genes, however, bloodstream levels of Aβ40 and Aβ42 are increased, whereas the soluble Aβ protein levels in the CSF (cerebrospinal fluid) of patients with AD decrease as compared to normal controls. Specifically, Aβ42 ranges in AD CSF are 110˜700 pg/ml; those with cognitively normal (CN) CSF are 245˜1600 pg/ml. Because Aβ levels in AD and non-AD groups have a high degree of overlap, measuring Aβ42 levels in the blood is used in laboratory research but not for clinical use. Further, even if the test of Aβ level were widely available, information to date indicates that blood Aβ levels would not be diagnostically accurate in most patients with non-familial or late-onset AD. Further, APP, the precursor protein of Aβ, is found to increase in the CSF of those with early onset AD. However, several large studies have shown that APP may not have diagnostic value.

The major component of amyloid plaques in AD brains is Aβ. β-secretase (BACE1) is one of the two key enzymes in processing the amyloid precursor protein (APP); the other being γ-secretase. BACE1 is a transmembrane aspartyl protease with all the known characteristics of APP β-secretase. Overexpression of this enzyme increases the amount of BACE1 cleavage products, C99 and C89. The role of BACE1 in Aβ production may explain the higher production of Aβ peptide in AD individuals and the early onset of Swedish familial Alzheimer's disease. To produce Aβ, APP must first be cleaved by BACE1 to produce a C99 fragment and release soluble APPβ (sAPPβ); C99 is then further cleaved by y-secretase to produce Aβ.

β-secretase (BACE) is a biomarker for MCI and Alzheimer's disease. BACE1 protein level and the enzymatic activity level increase in the brain, and in the CSF in AD and MCI patients. BACE1 protein level and the enzymatic activity level increase in Alzheimer APP23 transgenic mice overproducing Aβ and develop significant amyloid deposits. Increased BACE1 protein and enzymatic activity levels increase the cleavage product, C99, which can be further cleaved by γ-secretase to produce Aβ. Increased BACE1 protein and enzymatic activity levels are correlated to the increase of the total level of Aβ in AD and MCI patients. Increased BACE1 protein and enzymatic activity levels in CSF are also related to neuronal death, which can be quantified by the level of neuronal skeleton protein, tau. Increased BACE1 levels also correlate to synaptic damages, which contribute to AD-related cognitive deficits. For diseases including but not limited to AD, MCI, or BACE1-related diseases, BACE1 is not only a biomarker for diagnostic, or prognostic applications, but also a target for treatment or prevention of BACE1-related diseases, in which BACE1 protein level or enzymatic activity level is increased compared to normal control, and the increase of the BACE1 level contributes to the cause or the progression of the diseases. BACE1 inhibitors can be used as therapeutic compounds for clinical use for the disease treatment and prevention.

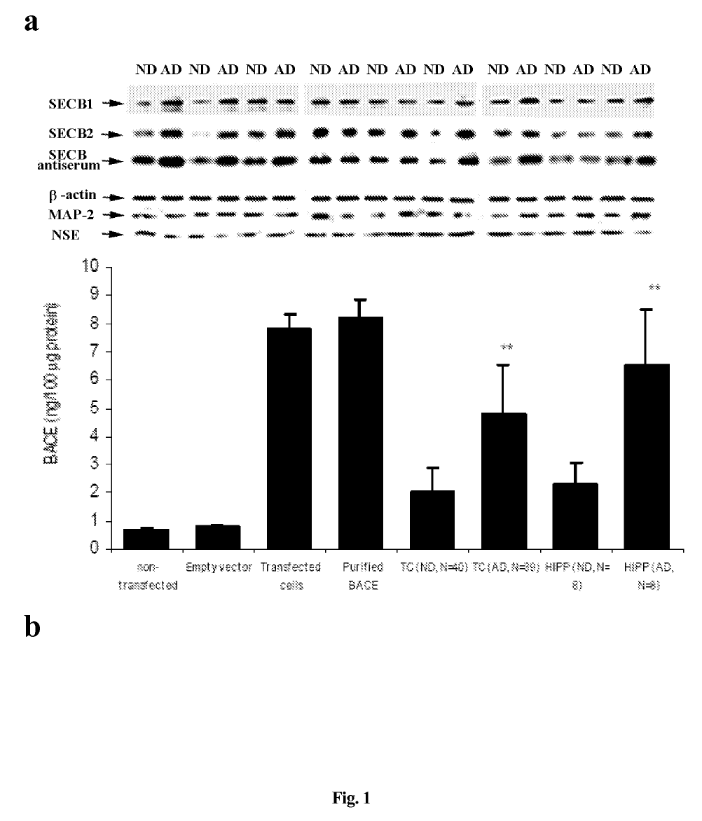

BACE1 protein level increased in brains of AD patients. Alzheimer's disease (AD) is the most common cause of dementia in the elderly population over 60 years of age. Lack of neuroplasticity, including synaptic deficits, senile plaques and paired helical filaments are the hallmarks of the brain pathology in AD. BACE1's protein level increased in vivo in rapidly autopsied brains of sporadic AD patients (<3 hours). Using western blot analysis, we found that BACE1 protein levels were higher in the temporal cortex of AD brain (n=18) than in brain from non-demented controls (ND; n=18) (FIG. 1a). An accurate ELISA assay was developed to test a comprehensive set of clinical samples. Temporal cortex and hippocampus samples were randomly selected from 39 clinically diagnosed and neuropathologically confirmed AD patients and 40 ND controls at the average ages of 84.5±6.12 and 84.0±8.12 years, respectively. The BACE1 ELISA further demonstrated that the BACE1 protein levels in sporadic AD brains are significantly higher compared to those found in normal aged controls (FIG. 1b).

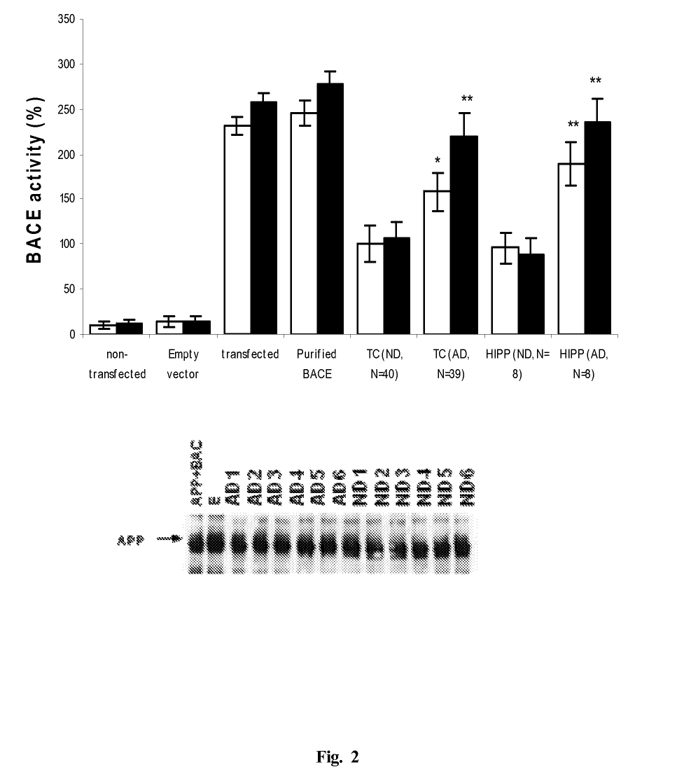

BACE1 enzymatic activity level increased in brains of AD patients. Fluorescent transfer peptides bearing the APP wild-type peptide (APPwt) BACE1 cleavage site (KTEEISEVKMDAE) or APP Swedish mutation (APPsw) BACE1 cleavage site (KTEEIVNLDAE) were used to test BACE1 enzymatic activity. These peptides were also labeled with methoxycoumarin acetic acid (MCA) and dinitrophenyl (DNP). As a positive control, we transiently transfected BACE1 cDNA or empty vector into HEK293 cells, and observed high-intensity fluorescence with BACE1-transfected cells (FIG. 2A). Enzymatic activity, represented by the fluorescent intensity of APPwt and APPsw fragments, increased in AD brain samples compared with ND samples (FIG. 2A). Further, more APPsw fragment than the APPwt fragment was cleaved by crude extracts from AD brain (FIG. 2A). To rule out possible involvement of caspases in AD brains, we incubated enzymatic crude extracts with a caspase inhibitor, ZVAD (Ome)-CH2F, which did not perturb activity in our assay. A plot of BACE1 activity versus BACE1 levels from ELISA data exhibited a distinct correlation between high BACE1 enzymatic activity level with high BACE1 protein levels in AD brains and low BACE1 enzymatic activity level with low BACE1 protein levels.

BACE1 cleavage product, C99, increased in brains of AD patients. Higher production of Aβ peptide is a hallmark in AD individuals and the early onset of Swedish familial Alzheimer's disease. To produce Aβ, APP must first be cleaved by BACE1 to produce a C99 fragment and release soluble APPβ (sAPPβ); C99 is then further cleaved by γ-secretase to produce Aβ. As BACE1 protein and enzymatic activity levels are increased in AD brains, to measure the level of C99, immunoprecipitated BACE1-cleaved C-terminal fragments using 4G8 antibody, which recognizes amino acids 17-24 of Aβ was applied. Followed by the western blot analysis with 6E10 antibody, more C99 fragments were immunoprecipitated to a higher level (P<0.01) in AD brains (FIG. 2B). This result is consistent with the above observations and further indicates that BACE1 activity in the AD brain is higher than that in ND.

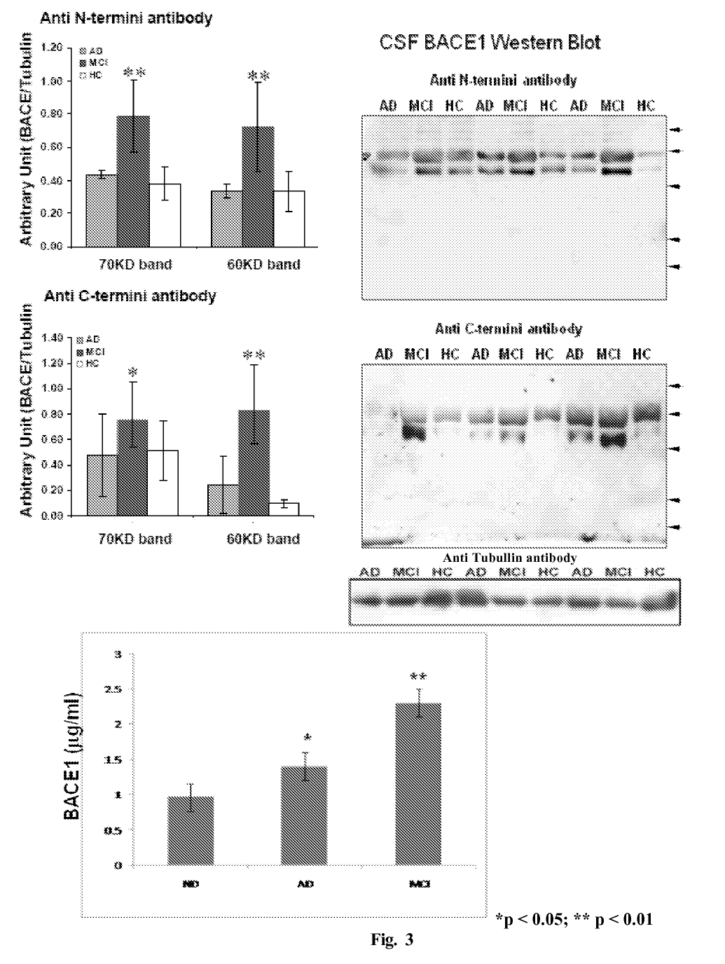

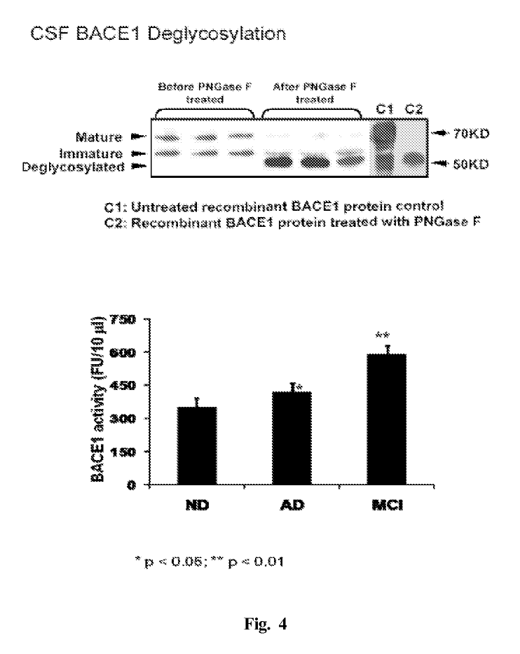

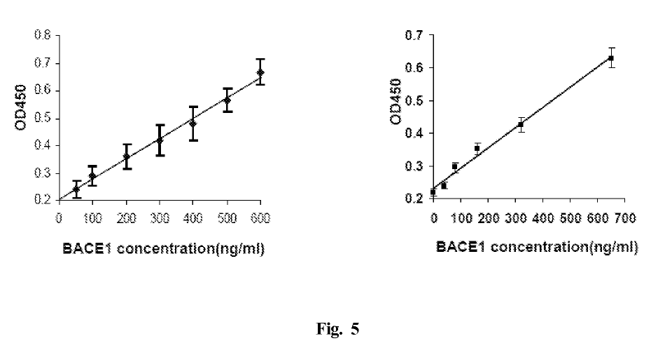

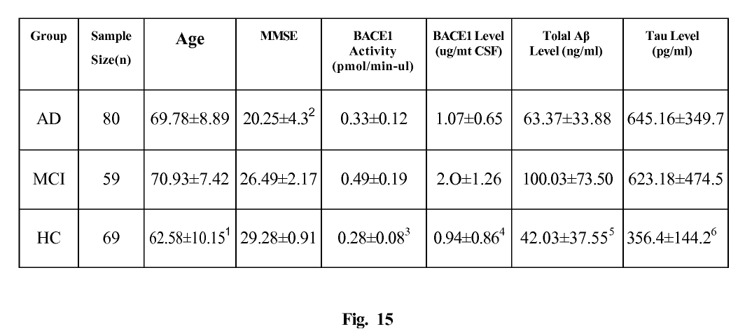

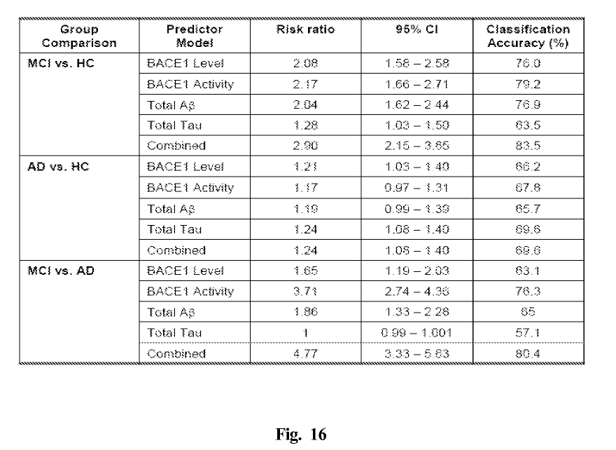

BACE1 protein level and enzymatic activity level increase in CSF of mild cognitive impairment (MCI) patients. To examine whether there was any BACE1 in the CSF of AD patients, Western Blotting was performed by using different antibodies against BACE1. When using the anti-N terminus antibody, both the 70 KD BACE1 and ˜60 KD BACE1 proteins were detected in all CSF groups (FIG. 3). To examine whether they were full-length BACE1 proteins, the antibody that recognizes the C-terminus of BACE1 was used. The same bands were detected (FIG. 3). Further, both the mature and immature forms of BACE1 protein exist in CSF as the deglycosylation test had shown (FIG. 4). Two sandwich enzyme-linked immunosorbent assays have been used to evaluate CSF BACE1 protein levels (FIG. 5). Means of synthetic fluorescence substrate and total Aβ peptide are used for measuring CSF BACE1 enzymatic activities. In comparison to normal controls and AD patients, increased levels of both BACE1 protein and BACE1 activity in CSF were found to associate with increased risk ratios for patients with MCI (FIG. 15 and FIG.16). CSF BACE1 levels and activity were altered in MCI subjects but not in AD patients when compared to CN individuals (FIG. 15 and FIG. 16). Thus, elevation of BACE1 protein level and enzymatic activity level in CSF is also an indicator of MCI. Early detection of any elevation in BACE1 levels aids in the prediction of AD in patients with MCI, or for those who show a high risk for developing AD. (FIG. 15 and FIG. 16). A small study also suggests that BACE1 activity in CSF is also increased in Creutzfeldt-Jakob disease (CJD).

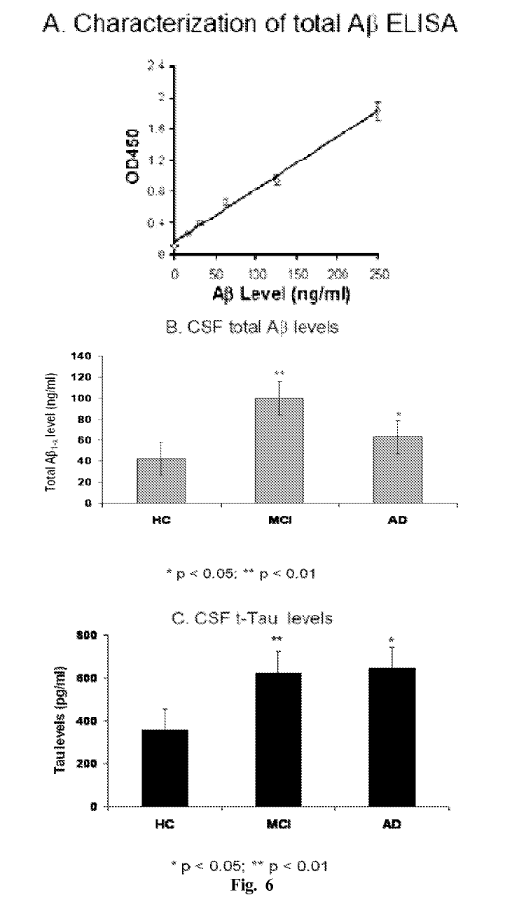

Increased levels of BACE1 correlate with increased CSF total Aβ levels in AD and MCI patients. CSF total Aβ levels were examined by sandwich ELISA (FIG. 6A). Differences in CSF total Aβ levels between AD, MCI and CN patient groups were statistically significant (F=11.94; df=2, 156, P<0.001) with a higher level in MCI patients compared to AD patients (P<0.001) and CN (P<0.001). The effect of diagnosis was not dependent on gender (P>0.05). No significant difference was observed between AD and CN (P>0.05; FIG. 6B). Increased Aβ levels were associated with a higher risk ratio to exhibit MCI compared to CN or AD. No elevated risk ratio associated with levels of Aβ was present when AD patients were compared with CN.

Increased BACE1 levels in CSF were related to neuronal death in AD and MCI patients. Neuronal skeleton protein tau was measured as a surrogate marker of neurodegeneration in CSF of AD and MCI patients. CSF total tau protein (t-tau) concentrations in AD and MCI patients were significantly higher than in CN patients (F=7.47; df=2, 146, P=0.001) (FIG. 6C), with the difference between AD and MCI not reaching statistical significance (P>0.05). The interaction between gender and diagnosis was not statistically significant (P>0.05; FIG. 6C, FIG. 15 and FIG. 16). Increased tau levels were associated with higher risk of AD compared to CN, and higher risk for MCI in comparison to CN as well. The level of tau between AD and MCI patients was not significant. (FIG. 6C, FIG. 15 and FIG. 16).

Transgenic mouse model is used for Alzheimer's disease research. Transgenic APP23 mice express a mutant APP that results in extensive Aβ plaque formation. Alzheimer's transgenic APP23 mice overproduce Aβ and develop significant amyloid deposits at about 12 months old. These models are used increasingly to assess novel AD biomarkers and treatments, particularly examining contributions of these molecules, including neuronal and synaptic proteins, calbindin, synaptophysin, to AD-related cognitive deficits and to unravel the pathways that disrupt synaptic functions in AD transgenic mice. The analysis of related transgenic mouse models is beginning to unravel the pathogenic importance of specific AD-associated molecules.

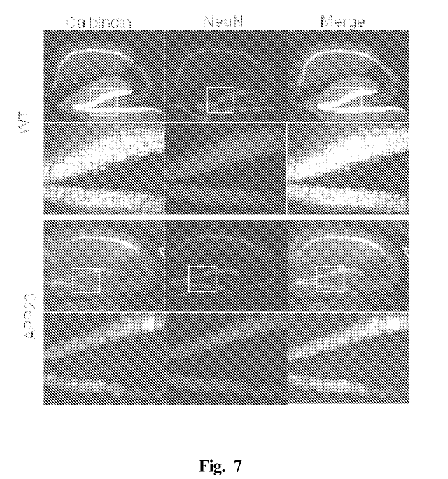

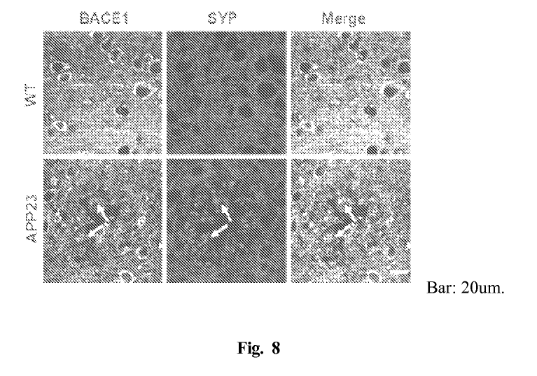

Synaptic damage exists in AD transgenic mouse model. Transgenic APP23 mice are used to assess novel AD biomarkers and treatments, particularly examining contributions of these molecules, including neuronal and synaptic proteins, calbindin (FIG. 7), and synaptophysin (FIG. 8), to AD-related cognitive deficits and to unravel the pathways that disrupt synaptic functions in AD transgenic mice. Calcium dependent protein, calbindin (CB) is abundant in hippocampal neurons, particularly in granule cells of the dentate gyms and pyramidal cells of the CA1 region. CB levels were significantly reduced in APP23 mice (FIG. 7), primarily in the granular layer of the dentate gyrus and in the molecular layer into which the granule cells extend their dendrites. Double-labeling of brain sections from APP23 mice for CB and the neuronal marker Neu-N (FIG. 7) indicated that the CB reductions in the dentate gyrus primarily reflected a decrease in neuronal CB levels rather than loss of CB-producing neurons.

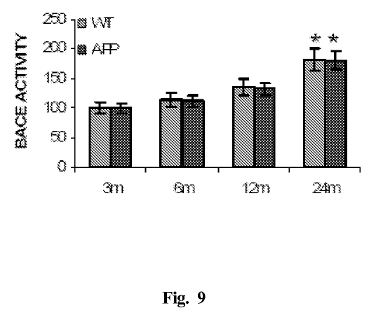

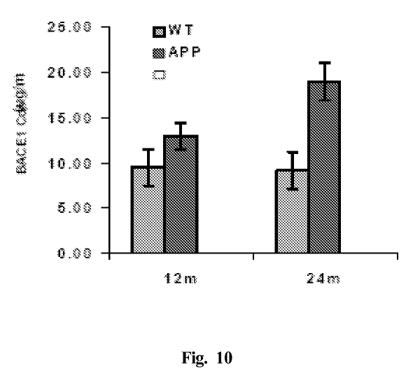

BACE1 enzymatic activity and protein levels increase in AD transgenic mouse model. BACE1 levels and activity are increased in postmortem brains of AD patients. Considering that numerous proteins become elevated in AD and the AD brain undergoes massive cell death, it is difficult to determine from postmortem brain whether any given change is an epiphenomenon in late-stage AD. However, BACE1 elevation could be recapitulated in APP23 transgenic mice, which develop amyloid plaques at young (12 months old) and old ages (24 months old), and it is suitable to examine whether BACE1 is an early event directly involved in pathogenesis. Both BACE1 enzymatic activity (FIG. 9) and protein levels are increased in brains of APP23 transgenic mice at 24 months (FIG. 10). BACE1 protein expression was increased significantly in brains of APP23 transgenic mice at both 12 months and 24 months in comparison to wildtype mice of the same age (FIG. 10).

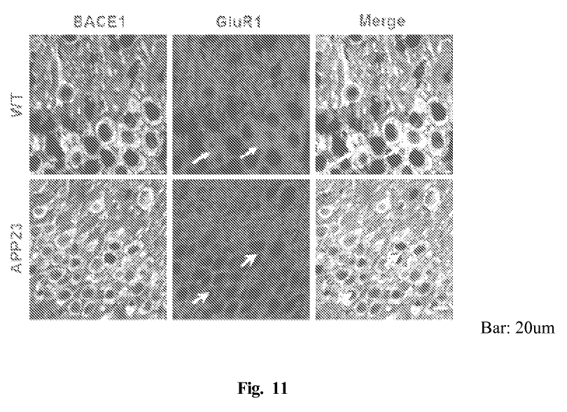

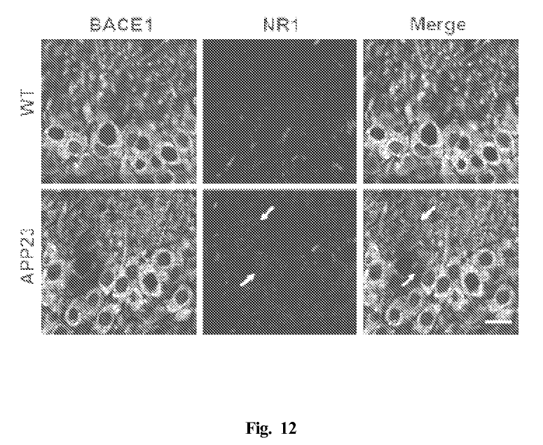

Increased BACE1 proteins and synaptic plasticity related proteins are expressed co-localized (in the same location). The elevation of BACE1 expression in the aged APP23 mice co-localizes with synapse related protein, glutamate AMPA receptor GluR1 (FIG. 11) as well as with NMDA receptor subtype, NR1 (FIG. 12). Glutamate receptors subtype, GluR1, and BACE1 primarily express on the cellular plasma membrane in the hoppocampal CA3 neurons (FIG. 11). GluR1 expression decreased in APP23 transgenic mice at 24 months, whereas the BACE1 expression on the CA3 neuron cell membrane increased, in comparison to wildtype mice of the same age (FIG. 11). Neuronal electrical activity stimulates BACE1 and therefore increases Aβ generation, and the resulting increased level of Aβ depresses synaptic transmission. With the result shown here, it is possible that BACE1 and soluble Aβ oligomers interfere with signaling pathways downstream of certain NMDA or AMPA receptors at synaptic plasma neuron cell membranes in a manner that allows an initial LTP (long term potentiation) response but not its persistence, and thus depresses synaptic transmission.

BACE1 inhibitors can be used as therapeutic compounds for clinical use. Considering the large and increasing number of AD cases and the devastating course of the disease, there is a great need for developing drugs that target critical pathologic mechanisms in AD. As a biomarker for AD and MCI, BACE1 activity can be measured in CSF. Therefore the upregulation of BACE1 may be an early pathogenic factor in AD, and is a target to develop method treating cognitive diseases including but not limited to, MCI, or Alzheimer' s disease.

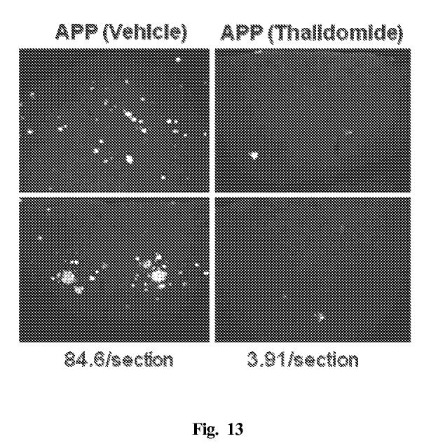

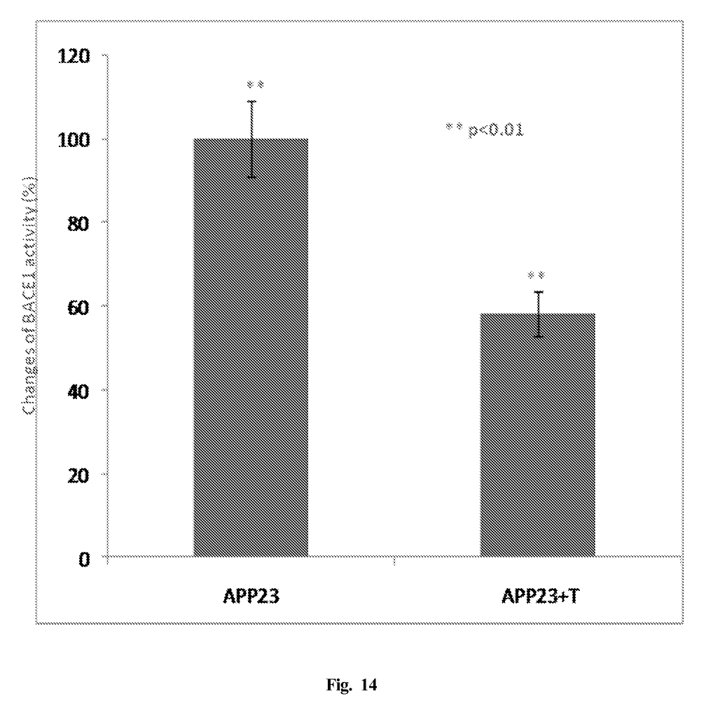

Thalidomide is an effective BACE1 inhibitor. The Thalidomide used to test effectiveness was ordered from Sigma, T144, 1.0 gram. The Thalidomide dose was 100 mg/kg/day. The application of Thalidomide to the mice born by the same mother was during the period from 9 months old to 12 months old. The preparation of Thalidomide was at first to be dissolved in DMSO and then diluted with saline. The injection into peritoneal cavity was performed once a day for 3 months. In general, the average weight of the mice was 30 grams. The volume of injection was 100 mg/kg/day×0.03 Kg/mouse=3 mg/mouse/day (if 10 mg/ml of Thalidomide, then 300 ul/mouse/day). After the treatment with Thalidomide, Alzheimer's transgenic mice APP23 had fewer Aβ plaques and Aβ-related lesions (FIG. 13). Detailed analyses showed reduced enzymatic activity level and protein level of BACE1 (FIG. 14), and decreased neuronal loss, and alleviated Aβ-related memory deficits. In both the in vitro and in vivo studies, Thalidomide resulted in a 42% decrease in BACE1 activity levels (FIG. 14), ultimately leading to a significant reduction of Aβ production and amyloid plaque numbers in Alzheimer's transgenic (APP23) mice (FIG. 13). The amyloid plaque number was 84.6/section in untreated control APP23 mice, where as there was only 3.91/section in the Thalidomide-treated APP23 mice. Use of Thalidomide in the transgenic mice reduced Aβ plaque formation and Aβ production, which is correlated to the reduced BACE1 enzymatic activity and protein level. Thus Thalidomide can decrease CSF BACE1 and CSF Aβ in AD and MCI patients. The inventor has initiated the clinical trial for the use of Thalidomide as BACE1 inhibitor in AD and MCI treatment.

Thalidomide is approved to treat multiple myeloma and erythema nodosum leprosum. It has been investigated to treat several different malignancies. The oncology literature reports doses administered up to 1200 mg/day, but maximum tolerable doses are between 400 and 800 mg/daily. The approved dose that is recommended by the FDA is 6.7 mg/m2. In a 70 kg person, the approved dose is approximately 450 mg. Therefore, 400 mg Thalidomide is suggested. This thalidomide regimen is tolerated by non-elderly subjects with other conditions without dose-related, severe or serious adverse events, and elderly subjects tolerate the medium dose at least as well as any dose administered to non-elderly patients.

Materials and Methods:

APP23 transgenic mice were provided by Novartis Institute for Biomedical Research; these mice express mutant human βAPP (Swedish double mutation, KM670/671NL) under the control of a brain- and neuron-specific murine Thy-1 promoter element. APP23 transgenic mice develop senile plaques in the cerebral cortex and hippocampus and show neuronal loss at 12-18 mo of age; this pathology is most evident in area CA1 of the hippocampus. APP23 mice were also constructed on a C57BL/6 background.

APP23 and wild-type mice (n=10 per group) were killed at 12 and 24 mo of age, and one hemisphere of the brain was homogenized in homogenization buffer (250 mM sucrose, 20 mM Tris-HCl, pH 7.4, 1 mM EDTA, and 1 mM EGTA). An aliquot of the homogenate was dissolved in formic acid and neutralized with a neutralization buffer (1 mM Tris and 0.5 M Na₂HPO₄). Protein concentration was measured by protein assay (Bio-Rad Laboratories). For total Aβ ELISA, the capture antibody was monoclonal anti-Aβ antibody 4G8 (Chemicon), and the detection antibody was biotinylated monoclonal antibody anti-Aβ 6E10 (AbD Serotec). Aβ40 and Aβ42 were measured with an Aβ40 and Aβ42 ELISA kit (Biosource International). The ELISA system has been extensively tested and no cross-reactivity between Aβ40 and Aβ42 was observed. Data are presented as means±SD of four experiments.

Western blotting was used to measure BACE1 protein level. Aliquots of brain homogenates from APP23 mice were further lysed with 1× RIPA buffer, and 50-150 μg of total protein was subjected to SDS-PAGE (8-12% acrylamide). Separated proteins were then transferred onto polyvinylidene fluoride membranes. The blots were probed with the following antibodies: anti-BACE1 monoclonal antibody (R&D Systems), anti-Aβ (1-17) monoclonal antibody (clone 6E10, 1:2,000; Chemicon), anti-IDE polyclonal antibody (Oncogene Research Products), anti-NEP polyclonal antibody (Chemicon), and anti—β actin antibody (Sigma-Aldrich).

To detect minute levels of Aβ using Western blotting, formic acid—dissolved brain tissue was immunoprecipitated with anti-Aβ polyclonal antibody (Zymed Laboratories) and subjected to SDS-PAGE using 10% acrylamide gels containing 8 M urea. Separated proteins were transferred onto polyvinylidene fluoride membranes. Aβ40 and Aβ42 were detected with monoclonal anti-Aβ antibody 6E10. Synthetic Aβ40 and Aβ42 (Biosource International) were used as standards.

BACE1 enzymatic activity was measured by using an aliquot of brain homogenate from APP23 and the wild-type mice. The aliquot was further lysed with a lysis buffer (10 mM Tris-HCl, pH 7.4, 150 mM NaCl, 1 mM EDTA, 1 mM EGTA, 1 mM Na₃VO₄, 10% glycerol, and 0.5% Triton X-100). BACE1 enzymatic activity assays were performed by using synthetic peptide substrates containing BACE1 cleavage site (MCA-Glu-Val-Lys-Met-Asp-Ala-Glu-Phe-[Lys-DNP]-OH; Biosource International). BACE substrate was dissolved in DMSO and mixed with a 50-mM Hac and 100-mM NaCl, pH 4.1, reaction buffer. An equal amount of protein was mixed with 100 μl of substrate, and fluorescence intensity was measured with a microplate reader (BioTek) at an excitation wavelength of 320 nm and an emission wavelength of 390 nm.

RT-PCR was used to compare BACE1 expression levels. The following primers were applied for RT-PCR: mouse BACE1 forward primer, 5′-AGACGCTACACATCCTGGTG-3′, and backward primer, 5′-CCTGGGTGTAGGGCACATAC-3′. The amplified BACE1 fragment was 146 bp. Mouse s18 was used as a loading control: forward primer, 5′-CAGAAGGACGTGAAGGATGG-3′, and backward primer, 5′-CAGTGGTCTTGGTGTGCTGA-3′. The amplified mouse s18 fragment was 159 bp. Total RNA was extracted from the brains of 12-mo-old APP23 and APP23/TNFR1^{-/-} mice (n=5) using an RNA mini column kit (Invitrogen). RT-PCR was performed using a One-Step RT-PCR kit (Invitrogen) and the following PCR cycles: 50° C. for 30 min, 94° C. for 2 min, followed by 25 cycles at 94° C. for 15 s, 49° C. for 30 s, and 68° C. for 1 min.

Immunohistochemistry and immunofluorescence Immunohistochemistry were performed, in which paraformaldehyde-fixed brains were quickly frozen, and then sectioned at 30 μm. Sections were incubated with either anti-Aβ (6E10 clone or 4G8 clone, 1:1,000; Chemicon), anti-NeuN (MAB377, 1:400; Chemicon), anti-CD11b (MCA711, 1:500; AbD Serotec) and CD45 (MCA1388, 1:500; AbD Serotec), anti-smooth muscle actin (-SM actin, A2547, 1:400; Sigma-Aldrich), or anti-vWF (AB7536, 1:200; Chemicon). Secondary antibodies were applied with horse anti-mouse (for 6E10, NeuN detection, 1:1,000) and goat anti-rat (for CD45 or CD11b, 1:1,000) followed by a DAB substrate (Vector Laboratories). For immunofluorescence, fluorescent-labeling 488 (green) or 594 (red) secondary antibodies against rabbit IgG or mouse IgG were used (1:1,000; Invitrogen). A microscope (DMLS; Leica) with a 10× N PLAN and 20× and 40× PL FLUOTAR was used. Digital images were captured and processed by digital camera (Optronics) and MagnaFire software (version 2.1C; Optronics).

Quantitation of immunoreactive structures 30-μm serial sagittal sections through the entire rostrocaudal extent of the hippocampus were cut on a cryostat. Every 10th section was immunostained with anti-NeuN antibody. On all sections containing the hippocampus, we delineated the pyramidal cell layer CA1. The total number of neurons was obtained using unbiased stereology and a microscope equipped with a digital camera (DEI-470; Optronics). For each section, we delineated a 400-μm² area in CA1 and in the entorhinal cortex and counted all NeuN-immunoreactive cells within that 400-μm² box. The mean sum of neurons was counted per animal (n=10). We used the same method to count Aβ-immunoreactive plaques (stained with 6E10) in the hippocampus and entorhinal cortex in a double blind test. We also measured the diameter of each counted plaque. Differences between groups were tested with Image-pro Plus Analysis (Media Cybernetics).

The Hole-board memory task was used to measure a mouse's ability to remember which one out of four equidistant holes was baited with food. Two photobeam apparatuses were used with a hole board for assessing directed exploration in mice for behavioral tests. A tested mouse (n=10 for each group) was placed in the center of the hole-board and the number of nose pokes was automatically registered for 5 min. After 20 min, each animal was placed in a corner of the hole board and allowed to freely explore the apparatus for 5 min. The number of head dips, time spent head-dipping, and the number of rearings was recorded. A comprehensive cognitive performance was determined by calculating the mean number of correct pokes per trial that mouse made each day. Cognition was expressed as the percentage of correct pokes. The measurements in the hole-board test were analyzed by unpaired t test. In all cases the significance level was considered to be P<0.05, and the very significant level was considered to be P<0.01.

Object recognition task was also applied to test the mice's level of cognition. The day before training, an individual mouse (n=10 for each group) was placed into a training apparatus (a box the same size as described for the hole-board test) and allowed to habituate to the environment for 15 min. Training was initiated 24 h after habituation. A mouse was placed back into the training box containing two identical objects A and B (die or marble) and allowed to explore these objects. Among experiments, training times varied from 3.5 to 20 min. For each experiment, the same set of animals was used repeatedly with different sets of objects for each repetition. Five repetitions were performed on each set of mice. Each mouse was trained and tested no more than once per week, with a 1-wk interval between testing. Moreover, each experimental condition was replicated independently four times. In each experiment, the experimenter was blinded to the subjects during training and testing. To test memory retention, mice were observed for 10 min, 6 h, and 24 h after training. Mice were presented with two objects, one that was used during training, and thus was “familiar,” and one that was novel. The test objects were divided into 10 sets of “training” plus “testing” objects, and a new set of objects was used for each training session. A recognition index was calculated for each mouse, expressed as the ratio (100TB)×(TA+TB), where TA and TB are the time spent during the second trial on subject A and subject B, respectively. To ensure that the discrimination targets did not differ in odor, the apparatus and the objects were thoroughly cleaned with 90% ethanol, dried, and ventilated for a few minutes after each experiment.

Statistical analysis of variance models (ANOVA) was used to analyze behavioral data. Typically, the statistical models included two between-subjects variables, the genotype of mice and age, and one within-subjects variable, such as blocks of trials. When ANOVAs with repeated measures were conducted, the Huynh-Feldt adjustment of levels was used for all within-subjects effects containing more than two levels to protect against violations of the sphericity/compound symmetry assumptions underlying this ANOVA model.

One BACE1 ELISA used a combination of a highly specific anti-BACE1 polyclonal antibody SECB2 as a capture antibody and biotinylated anti-BACE1 polyclonal antibody SECB1 as a detection antibody. The other ELISA was established by using anti-BACE1 polyclonal antibody B280 as a capture antibody and anti-BACE1 monoclonal antibody (R&D) as a detection antibody. Purified BACE1 from BACE1 transfected cells will be used as the standard and will be assayed under the same conditions. The concentration of BACE1 will be calculated from the standard curve and expressed as μg/ml.

BACE1 Enzymatic Activity Assay was performed by using synthetic peptide substrates containing the BACE1 cleavage site (MCA-Glu-Val-Lys-Val-Asp-Ala-Glu-Phe-(Lys-DNP)-OH in reaction buffer (50 mM acetic acid pH4.1, 100 mM NaCl). From each sample, 10 μl of CSF will be used to examine BACE1 activity. Fluorescence will be observed with a fluorescent microplate reader with an excitation wavelength at 320 nm and emission wavelength at 383 nm.

Total Aβ1-x ELISA was used to measure the total Aβ levels in plasma, carried out with anti-Aβ monoclonal antibody 4G8 (aa17-24) as a capture antibody and biotinylated anti-Aβ monoclonal antibody 7N22 as the detection antibody, whose epitope has been mapped to aa8-13. No cross-reactivity has been observed with Aβx-40 or Aβx-42, and we will use HPLC-purified Aβ1-x as a standard. Intra-assay variability is less than 3% (n=500); inter-assay variability is less than 10% (n=24). Sensitivity for the ELISA is less than 10 μg/ml. The assay is linear in the range of 0-1,000 pg/ml. Monoclonal antibody will be coated onto microtiter plates, and left overnight at 4° C. After washing the block plates at 25° C. with PBS plus tween, we will incubate alkaline phosphatase antibody simultaneously with plasma samples for 60 minutes. The amount of bound antibody will be determined by adding fluorescent substrate reagent for 30 minutes. All analyses will be run with the same batch of antibodies and ELISA plates.

t-tau ELISA: Tau was measured in duplicates with commercially available enzyme-immunosorbent assays (ELISA) (Innotest hTAU-Ag, Innogenetics, Zwjindrecht, Belgium, Art. No. K-1080 and Art No. K-1032). Data were expressed as mean±standard deviation.

To measure the Aβ deposition level in the brain of APP23 mice under the impact of Thalidomide, the mice (n=6) from the same mother were injected with Thalidomide for one month at a dosage of 100 mg/kg. The Aβ plaque number was determined via 6E10 immunostaining in the APP23 mice, and compared to the APP23 mice without Thalidomide treatment in the frontal cortex at the same age.

To evaluate the BACE1 enzymatic activity level under the impact of Thalidomide, the frontal cortices of 12-months old APP23 transgenic mice after Thalidomide treatment with doses of 100 mg/kg for one month were obtained for the enzymatic crude extracts. The extracts were then incubated with fluorescent-labeled peptide-bearing β-sites from APP protein (APPwt) or APP with the Swedish mutation (APPsw). Purified BACE1 or BACE1 transfected cell lysates were used as positive controls. The specific fluorescence was detected when labeled APPwt or APPsw peptides were cleaved by BACE1. The level of the fluorescence detected was used as measurement of the BACE1 enzymatic activity level.

We claim:

1. A method for the treatment of cognitive deficit diseases in a mammal, comprising the step of administering a therapeutically effective amount of Thalidomide or a pharmaceutically acceptable prodrug, salt, solvate, hydrate, or clathrate thereof to the mammal.

2. The method of claim 1 in which the mammal is a human.

3. The method of claim 1 in which the cognitive deficit disease to be treated is BACE1-related.

4. The method of claim 3 in which the cognitive deficit disease is a degenerative neurological disorder.

5. The method of claim 1 in which the cognitive deficit disease to be treated is Alzheimer's disease.

6. The method of claim 1 in which the cognitive deficit disease to be treated is Alzheimer's related dementia.

7. The method of claim 1 in which the cognitive deficit disease to be treated is Mild Cognitive Impairment.

8. The method of claim 1 in which the Thalidomide has the effect of inhibiting BACE1.

9. A kit used to treat cognitive deficit diseases comprising pharmaceutical composition and dosage form of Thalidomide or a pharmaceutically acceptable prodrug, salt, solvate, hydrate, or clathrate thereof.

10. A method for the prevention of Alzheimer's disease in a mammal comprising administering an effective amount of Thalidomide or a pharmaceutically acceptable prodrug, salt, solvate, hydrate, or clathrate thereof to the mammal.

11. The method of claim 10 in which the mammal is a human.

12. The method of claim 10 in which the mammal has Mild Cognitive Impairment.

13. The method of claim 10 in which the mammal has Alzheimer's related dementia.

14. The method of claim 10 in which the mammal has increased BACE1 protein level.

15. The method of claim 10 in which the mammal has increased BACE1 enzymatic activity level.

16. The method of claim 10 in which the Thalidomide has the effect of inhibiting BACE1;

17. The method of claim 10 in which the prevention is through slowing the loss of neuroplasticity.
